(19) United States
(12) Patent Application Publication
(10) Pub. No.: US 2022/0175732 A1
(43) Pub. Date: Jun. 9, 2022

(54) TREATMENT OF ROSACEA WITH TOPICAL COMBINATION COMPOSITIONS

(71) Applicant: Sol-Gel Technologies Ltd., Ness Ziona (IL)

(72) Inventor: Moshe ARKIN, Kfar Shmaryahu (IL)

(73) Assignee: Sol-Gel Technologies Ltd., Ness Ziona (IL)

(21) Appl. No.: 17/440,778

(22) PCT Filed: Mar. 19, 2020
(86) PCT No.: PCT/IL2020/050334

§ 371 Date: Sep. 19, 2021

Related U.S. Application Data
(60) Provisional application No. 62/820,498, filed on Mar. 19, 2019.

(51) Int. Cl. A61K 31/4164; A61K 9/00; A61K 31/7048; A61K 31/215; A61K 31/167; A61K 31/327; A61K 31/05; A61K 31/65; A61K 31/194; A61K 31/137; A61K 31/498; A61K 9/06; A61K 47/10; A61K 47/26; A61K 47/14; A61K 47/18; A61P 17/00

(52) U.S. Cl. CPC A61K 31/4164; A61P 17/00; A61K 31/7048; A61K 31/215; A61K 31/167; A61K 31/327; A61K 31/05; A61K 31/65; A61K 31/194; A61K 31/137; A61K 31/498; A61K 9/06; A61K 47/10; A61K 47/26; A61K 47/14; A61K 47/18; A61K 9/0014

(57) ABSTRACT

Provided herein is a topical combination composition comprising a therapeutically effective amount of at least one acaricidal active agent, a therapeutically effective amount of at least one essentially non-acaricidal anti-inflammatory active agent and a carrier suitable for topical administration for the treatment of rosacea. Also provided are combination compositions optionally further comprising a vasoconstrictor.

### FIELD OF THE INVENTION

The present invention, in some embodiments thereof, relates to pharmaceutical topical combination compositions for the treatment of rosacea, comprising active agents from two or more different classes, more particularly to compositions comprising at least one acaricidal active agent and at least one essentially non-acaricidal anti-inflammatory active agent.

The compositions of this invention are useful for the treatment, prevention or amelioration of rosacea and exhibit additive and/or synergistic effects.

### BACKGROUND

Rosacea is a chronic disease of inflammatory dermatitis that mainly affects the median part of the face and the eyelids of certain adults. It is characterized by telangiectatic erythema, dryness of the skin, papules and pustules. Typically, rosacea develops in adults from the ages of 30 to 50; it is more frequently affects women, although the condition is generally more severe in men. Rosacea is a primarily vascular condition whose inflammatory stage lacks the cysts and comedones characteristic of common acne.

Factors that have been described as possibly contributing towards the development of rosacea include for example: parasites such as the Demodex folliculorum, bacteria such as Helicobacter pylori (a bacterium associated with gastrointestinal disorders), hormonal factors (such as endocrine factors), climatic and immunological factors, and so forth.

Rosacea develops in four stages over several years, in spasms aggravated by variations in temperature, alcohol, spices, exposure to sunlight and stress.

The various stages of the disease are the following:

Stage 1: stage of erythema episodes. The patients have erythrosis spasms due to the sudden dilation of the arterioles of the face, which then take on a congestive, red appearance. These spasms are caused by the emotions, meals and temperature changes.

Stage 2: stage of couperosis, i.e., of permanent erythema with telangiectasia. Certain patients also have oedema on the cheeks and the forehead.

Stage 3: inflammatory stage (papular-pustular rosacea) with appearance of inflammatory papules and pustules, but without affecting the sebaceous follicles and thus with absence of cysts and comedones.

Stage 4: rhinophyma stage. This late phase essentially affects men. The patients have a bumpy, voluminous red nose with sebaceous hyperplasia and fibrous reordering of the connective tissue.

Typical treatments of rosacea include oral or topical administration of antibiotics such as tetracyclines, salicylic acid, anti-fungal agents, steroids, metronidazole (an anti-bacterial agent), isotretinoin in severe cases, or anti-infectious agents such as azelaic acid.

US 20110052515 describes a topically applicable formulation for treating rosacea, comprising at least one avermectin compound and benzoyl peroxide (BPO, an anti-acne agent).

Montes et al. (Cutis, 32, 185-190 (1983)) disclosed the use of BPO dissolved in acetone gel formulation for the treatment of rosacea.

Considering the chronic nature of rosacea, there is a need for a prolonged use treatment of the disease, its symptoms and associated conditions, in a safe and effective manner. Thus, there exists an unmet need for a novel approach in rosacea treatment, enabling improved efficacy in the treatment of rosacea, thus making possible the long-term treatment of this chronic disease.

### SUMMARY OF THE INVENTION

This invention provides a combination composition comprising at least one acaricidal active agent, and at least one essentially non-acaricidal anti-inflammatory active agent.

The active agents in the combination composition are from different classes. While the at least one acaricidal active agent in the combination composition of this invention acts against the mites Demodex folliculorum, the at least one essentially non-acaricidal anti-inflammatory active agent acts against the rosacea in its inflammatory stage.

The combination compositions may further comprise a vasoconstrictor.

The above compositions are useful for the treatment, prevention or alleviation of rosacea by topical administration to a subject in need thereof a therapeutically effective amount of the composition of this invention.

### DETAILED DESCRIPTION OF THE INVENTION

This invention provides a novel approach for the topical treatment of rosacea, based on the understanding that this disease has several different phases.

Treating rosacea disease with a composition combining drugs from two or more different classes, (i) an acaricide, (ii) an essentially non-acaricidal anti-inflammatory active agent and (iii) optionally a vasoconstrictor; is more effective than treating rosacea with the individual single drugs or with combinations of drugs from the same class, affording higher clearance rates and/or longer remission periods between clearance and reappearance of rosacea.

More particularly, this invention provides a combination composition comprising at least one acaricidal active agent, at least one essentially non-acaricidal anti-inflammatory active agent and optionally a vasoconstrictor.

The at least one acaricidal active agent in the combination composition of this invention kills the mites Demodex folliculorum (now recognized as one of the main rosacea causative factors) and the at least one essentially non-acaricidal anti-inflammatory active agent treats rosacea in the inflammatory stage. The vasoconstrictor counters the effect of erythrosis spasms due to the sudden dilation of the arterioles of the face. Moreover, as most of the selected anti-inflammatory active agents (vide infra) are also anti-infectious, antibiotic, antibacterial and/or anti-fungal, these active agents provide an additional therapeutic angle to the combination compositions of this invention.

The combination compositions of this invention are useful for the treatment, prevention or alleviation of rosacea by topical administration to the rosacea affected skin area of a subject in need thereof a therapeutically effective amount of said combination compositions. The combination compositions exhibit synergistic and/or additive effects, thus allowing to reduce the amounts of the active agents in the compositions.

The combination of active agents from two or more different classes is more effective than the individual single drugs, with higher clearance rates and/or longer remission periods between clearance and reappearance of rosacea.

In some embodiments, there is provided a topical composition comprising a therapeutically effective amount of at least one acaricidal active agent, a therapeutically effective amount of at least one essentially non-acaricidal anti-inflammatory active agent, optionally at least one vasoconstrictor and a carrier suitable for topical administration. The acaricidal active agent in the above composition is selected from ivermectin, permethrin, niclosamide, benzoyl peroxide, tapinarof and combinations thereof. The essentially non-acaricidal anti-inflammatory active agent in the above composition is selected from a tetracycline, minocycline, doxycycline, metronidazole, azelaic acid and combinations thereof.

Each combination of at least one acaricidal active agent and at least one essentially non-acaricidal anti-inflammatory active agent is a separate embodiment.

### Acaricidal Active Agents

Acaricides are pesticides that kill members of the arachnid subclass of Acari parasites. Acari includes mites and ticks. Mites and ticks are usually treated as a single group. A less common name is “miticides” which are substances killing mites.

The purpose of the at least one acaricide component of the composition is to kill the mites of the Demodex folliculorum, which is one of the causes of rosacea.

A number of acaricides are commercially available, the most prominent being permethrin, ivermectin and crotamiton.

For the purpose of this invention, acaricides (miticides) potentially active against Demodex folliculorum are selected. The at least one acaricidal active agent in the composition of this invention is selected from ivermectin, permethrin, niclosamide, benzoyl peroxide, tapinarof and combinations thereof.

Tapinarof is a first-in-class drug in development, which showed promising results in the topical treatment of psoriasis and also activity as acaricide.

Tapinarof (3,5-dihydroxy-4-isopropyl-trans-stilbene, benvitimod, GSK2894512) is a first-in-class drug, whose mechanism is not yet fully understood. It is being developed by Glaxo Smith Kline (Stiefel, a GSK company) and Dermavant as a topical drug for treatment of mild to moderate plaque psoriasis and atopic dermatitis. It was shown in both mouse models and in vitro human skin studies to inhibit specific proinflammatory mediators, including interleukin-6 and interleukin-17A, and enhance skin barrier function (J Invest Dermatol. 2017 October; 137[10]:2110-9).

Topical benzoyl peroxide is presently used mainly for acne vulgaris treatment.

### Non-Acaricidal Anti-Inflammatory Active Agents

The anti-inflammatory active agents selected for the composition of this invention are a tetracycline, minocycline, doxycycline, metronidazole, azelaic acid and combinations thereof. Most of the above anti-inflammatory active agents are also anti-infectious, antibiotic, antibacterial and/or anti-fungal and this is an additional therapeutic advantage of the combination compositions of this invention. The selected anti-inflammatory active agents are essentially non-acaricidal.

### Topical Combination Compositions Further Comprising a Vasoconstrictor in Addition to at Least One Acaricidal Active Agent and at Least One Essentially Non-Acaricidal Anti-Inflammatory Active Agent

The combination compositions of this invention may optionally further comprise a vasoconstrictor in addition to at least one acaricidal active agent and at least one essentially non-acaricidal anti-inflammatory active agent, wherein the vasoconstrictor is optionally encapsulated. The vasoconstrictor is selected from phenylephrine, oxymetazoline, brimonidine, a NOS inhibitor, tetrahydrozoline, pseudoephedrine and combinations thereof.

### Topical Combination Compositions

Provided herein are compositions, methods, combinations, kits and articles of manufacture comprising at least one acaricidal active agent selected from ivermectin, permethrin, niclosamide, benzoyl peroxide, tapinarof, at least one non-acaricidal active agent selected from a tetracycline, minocycline, doxycycline, metronidazole, azelaic acid and combinations thereof and optionally a vasoconstrictor selected from phenylephrine, oxymetazoline, brimonidine, a NOS inhibitor, tetrahydrozoline, pseudoephedrine and combinations thereof, for treating rosacea.

The selected at least one acaricidal active agent, the selected at least one non-acaricidal active agent and the selected vasoconstrictor may be combined in various ways, providing a large number of possible combinations. Each combination is a separate embodiment.

### Exemplary Combination Compositions

Several exemplary combination compositions are detailed in the following. Each of the combinations below is a separate embodiment. Some of these compositions are described in Examples 1-2.

1) A combination composition comprising from about 1% to about 12% benzoyl peroxide and from about 1% to about 8% w/w minocycline HCl. This composition presents several problems: a. benzoyl peroxide is a strong oxidant, which may compromise the chemical stability of minocycline and b minocycline is sensitive to water which, again, may compromise the chemical stability of minocycline. This precludes compositions comprising water, like creams for example. The solution proposed is to apply topically the composition of this invention to the affected skin area of a subject in need thereof (simultaneously or sequentially in either order) as two separate compositions to be rejoined on the subject's skin, the first composition comprising from about 1% to about 12% benzoyl peroxide and a carrier suitable for topical administration and the second composition comprising from about 1% to about 8% w/w minocycline and a carrier suitable for topical administration. This can be done for example by applying the two separate compositions to the affected area of the skin of a subject in need thereof from two application syringes or from a dual chamber application syringe, simultaneously or sequentially in either order. The preferred concentrations of the two compositions are 12% BPO and 4% minocycline HCl.

In some embodiments, the minocycline composition comprises 1.0%-8.0% w/w minocycline HCl, 4%-5% w/w cyclomethicone, 21%-27% coconut oil, 46%-54% w/w soybean oil. 2%-3% miristyl alcohol, 1%-1.5% w/w behenyl alcohol, 3.0%-20% w/w cetyl alcohol, 0.25% w/w silicone dioxide.

In a preferred product according to the invention, first and second formulations are respectively presented in the chambers of a dual chamber dispense system of the type described in EP-A-0644129 and U.S. Pat. No. 5,356,040, the contents of which are incorporated herein by reference. Such a system has two side-by-side chambers, each equipped with a dispense valve; these are operated by adjacent actuators so as to dispense the formulations either simultaneously or separately as desired. Suitable dispense systems, e. g. having chambers which are each capable of holding about 15 ml of formulation, are available from Maplast S. r. 1., Via Pasublo 3, Tradate 21049 VA, Italy. The respective dimensions of the dispense means may be chosen to provide dispense of the respective formulations in a predetermined ratio.

2) A combination composition comprising from about 0.1% w/w to about 2.0% w/w tapinarof and from about 0.25% w/w to about 2.0% w/w metronidazole (Example 1).

3) A combination composition comprising from about 1% w/w to about 10.0% w/w benzoyl peroxide and from about 10.0% w/w to about 16.0% w/w azelaic acid.

4) A combination composition comprising from about 1% w/w to about 6.0% w/w permethrin and from about 10.0% w/w to about 16.0% w/w azelaic acid.

5) A combination composition comprising from about 0.1% w/w to about 2.0% w/w tapinarof, from about 0.25% w/w to about 2.0% w/w metronidazole and from about 0.1% w/w to about 0.5% w/w phenylephrine, wherein phenylephrine is optionally encapsulated (Example 2).

The compositions, combinations and articles of manufacture of this invention can be administered using a variety of routes such as topical application or transdermal application. The preferred route is the topical route and the preferred formulations are the cream, the lotion, the gel and the foam.

The active agents in the combination compositions are included in an amount effective for treating, preventing or reducing the rosacea symptoms. The concentration of the active agents in the composition will depend on absorption, inactivation, excretion rates of the active agent, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of same active agents in the marketed single drug currently administered or being developed for the treatment of rosacea. The dosage and regimen of administration may be determined by dose finding studies, as known in the art.

Exemplary dosages, strengths and concentrations of tapinarof in the tapinarof combination compositions administered topically, can be 0.1%, 0.25%, 0.5%, 1%, 2% or 3% w/w. Typical strengths in the topical combination compositions of this invention are 0.1%, 0.25%, 0.5% or 1% w/w tapinarof. The frequency of administration can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the rosacea. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams or any other formulation suitable for topical administration. The preferred compositions are the cream, the lotion, the gel and the foam.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of rosacea, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, foams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Methods of Treatment

According to an aspect of the invention, there is provided a method of treatment of rosacea by treatment of a subject in need thereof with a combination composition comprising at least one acaricide, at least one essentially non-acaricidal anti-inflammatory active agent and optionally a vasoconstrictor.

In some embodiments, the effective amount is a therapeutically effective amount of the active agents, namely an amount which will cure, treat, mitigate or prevent rosacea.

In some embodiments, co-administration of the active agents exhibits an additive and/or synergistic effect while treating, preventing or alleviating rosacea.

In some other embodiments, the co-administration may be made either by administration of a single combination composition, or alternatively by separate administration of a first composition comprising at least one acaricide and a second composition comprising at least one essentially non-acaricidal active agent.

In some other embodiments, the co-administration may be made either by administration of a single combination composition, or alternatively by separate administration of a first composition comprising at least one acaricide and a second composition comprising at least one essentially non-acaricidal active agent and optionally a third composition comprising a vasoconstrictor.

### Regimen of Administration of the Topical Rosacea Combination Compositions

Therapeutically effective concentrations of active agents in the compositions of this invention for treatment, prevention or amelioration of the symptoms manifested by rosacea are determined by empirical methods known in the art.

The concentration of the active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of the same active agents in the developed or marketed single drug currently being developed or used for the treatment of rosacea. The dosage and regimen of administration may be determined by dose finding studies, as known in the art.

Exemplary dosages, strengths and concentrations of tapinarof in the tapinarof combination compositions administered topically, can be in the range of from about or at 0.1%, 0.25%, 0.5%, 1%, 2% or 3% w/w.

Typical strengths in the topical combination compositions of this invention are 0.1%, 0.25%, 0.5% or 1% w/w tapinarof.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

In some other embodiments, compositions are administered by separate administration of a first composition comprising at least one acaricide and a second composition comprising at least one essentially non-acaricidal active agent and optionally a third composition comprising a vasoconstrictor; wherein the compositions are administered, concomitantly (same time) once daily or twice daily from a dual chamber dispenser or from separate dispensers and mixed before applying on the skin of a patient in need thereof.

In another embodiment, the vasoconstrictor may be combined in one embodiment with the first composition comprising at least one acaricide. In another embodiment, the vasoconstrictor may be combined in one embodiment with the second composition comprising at least one essentially non-acaricidal active agent.

### Kits

Kits containing the combination compositions optionally including instructions for administration are provided. The combinations include, for example, the compositions as provided herein. Additionally, provided herein are kits containing the above-described combinations and optionally instructions for administration by topical, transdermal, or other routes, depending on the composition to be delivered.

The compositions provided herein can be packaged as articles of manufacture containing packaging material, a composition provided herein, and a label that indicates that the composition is for treating rosacea, and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Embodiments

In some embodiments, there is provided a topical composition comprising a therapeutically effective amount of at least one acaricidal active agent, a therapeutically effective amount of at least one essentially non-acaricidal anti-inflammatory active agent and a carrier suitable for topical administration.

In some other embodiments, there is provided a topical composition comprising a therapeutically effective amount of at least one acaricidal active agent, a therapeutically effective amount of at least one essentially non-acaricidal anti-inflammatory active agent and a carrier suitable for topical administration, wherein the acaricidal active agent is selected from ivermectin, permethrin, niclosamide, benzoyl peroxide, tapinarof and combinations thereof.

According to some embodiments, there is provided a topical composition comprising a therapeutically effective amount of at least one acaricidal active agent, a therapeutically effective amount of at least one essentially non-acaricidal anti-inflammatory active agent and a carrier suitable for topical administration, wherein the essentially non-acaricidal anti-inflammatory active agent is selected from a tetracycline, minocycline, doxycycline, metronidazole, azelaic acid and combinations thereof.

According to some other embodiments, there is provided a topical composition comprising from about 1% to about 12% benzoyl peroxide, from about 1% to about 8% w/w minocycline and a carrier suitable for topical administration, wherein said composition is optionally formulated as two separate compositions, the first composition comprising from about 1% to about 12% benzoyl peroxide and a carrier suitable for topical administration and the second composition comprising from about 1% to about 8% w/w minocycline and a carrier suitable for topical administration.

In some embodiments, there is provided a topical composition comprising from about 0.1% w/w to about 2.0% w/w tapinarof, from about 0.25% w/w to about 2.0% w/w metronidazole and a carrier suitable for topical administration.

In some other embodiments, there is provided a topical composition comprising, from about 1% w/w to about 10.0% w/w benzoyl peroxide, from about 10.0% w/w to about 16.0% w/w azelaic acid and a carrier suitable for topical administration.

According to some embodiments, there is provided a topical composition comprising from about 1% w/w to about 6.0% w/w permethrin, from about 10.0% w/w to about 16.0% w/w azelaic acid and a carrier suitable for topical administration.

According to some embodiments, there is provided any of the above topical compositions of this invention, further comprising a vasoconstrictor selected from phenylephrine, oxymetazoline, brimonidine, a NOS inhibitor, tetrahydrozoline, pseudoephedrine and combinations thereof, wherein the vasoconstrictor is optionally encapsulated.

According to some other embodiments, there is provided a dosage form comprising a composition comprising a therapeutically effective amount of at least one acaricidal active agent, a therapeutically effective amount of at least one essentially non-acaricidal anti-inflammatory active agent and a carrier suitable for topical administration, wherein said composition is formulated in a dosage form selected from a cream, a lotion, a gel, an ointment, an emulsion, a solution, a suspension, an elixir, a tincture, a paste, a foam, an aerosol, a spray, a patch, a transdermal patch and an application syringe.

In some embodiments, there is provided a method of treatment, prevention or alleviation of rosacea by topical administration to the affected skin area of a subject in need thereof a therapeutically effective amount of a composition comprising at least one acaricidal active agent selected from ivermectin, permethrin, niclosamide, benzoyl peroxide, tapinarof and combinations thereof, at least one non-acaricidal active agent selected from a tetracycline, minocycline, doxycycline, metronidazole, azelaic acid and combinations thereof, optionally a vasoconstrictor selected from phenylephrine, oxymetazoline, brimonidine, a NOS inhibitor, tetrahydrozoline, pseudoephedrine and combinations thereof and a carrier suitable for topical administration,

In some other embodiments there is provided the above method of treatment, wherein the treatment comprises once daily or twice daily topical administration to the affected skin area of a subject in need thereof of a therapeutically effective amount of said composition.

In some embodiments, there is provided a method of treatment wherein the treatment comprises topical administration to the affected skin area of a subject in need thereof of a therapeutically effective amount of a composition comprising from about 1% to about 12% benzoyl peroxide, from about 1% to about 8% w/w minocycline and a carrier suitable for topical administration, wherein said composition is optionally formulated as two separate compositions, the first composition comprising from about 1% to about 12% benzoyl peroxide and a carrier suitable for topical administration and the second composition comprising from about 1% to about 8% w/w minocycline and a carrier suitable for topical administration and wherein said two separate compositions are administered from two application syringes or a dual chamber application syringe, simultaneously or sequentially in either order.

According to some embodiments, there is provided a method of treatment, prevention or alleviation of rosacea by topical administration to the affected skin area of a subject in need thereof a composition comprising a therapeutically effective amount of at least one acaricidal active agent, a therapeutically effective amount of at least one essentially non-acaricidal anti-inflammatory active agent, optionally a therapeutically effective mount of a vasoconstrictor and a carrier suitable for topical administration, wherein the at least one acaricidal active agent and the at least one essentially non-acaricidal anti-inflammatory active agent in said composition exhibit an additive or synergistic effect, thereby allowing to reduce the amounts of the active agents in the composition.

According to some other embodiments, there is provided a regimen of administration comprising once daily or twice daily administration to the rosacea affected skin area of a subject in need thereof a dosage form comprising a therapeutically effective amount of at least one acaricidal active agent, a therapeutically effective amount of at least one essentially non-acaricidal anti-inflammatory active agent, optionally a vasoconstrictor and a carrier suitable for topical administration, wherein said composition is formulated in a dosage form selected from a cream, a lotion, a gel, an ointment, an emulsion, a solution, a suspension, an elixir, a tincture, a paste, a foam, an aerosol, a spray, a patch, a transdermal patch and an application syringe, until remission.

In some embodiments, there is provided a kit comprising one or more dosage forms comprising a therapeutically effective amount of at least one acaricidal active agent, a therapeutically effective amount of at least one essentially non-acaricidal anti-inflammatory active agent, optionally a therapeutically effective amount of a vasoconstrictor and a carrier suitable for topical administration, wherein said composition is formulated in a dosage form selected from a cream, a lotion, a gel, an ointment, an emulsion, a solution, a suspension, an elixir, a tincture, a paste, a foam, an aerosol, a spray, a patch, a transdermal patch and an application syringe and instructions for use.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

### Preparation of a Tapinarof-Metronidazole Cream Composition

The topical tapinarof-metronidazole combination cream consists of:

0.1%-2.0% w/w tapinarof,

0.25%-2.0% w/w metronidazole

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH,

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, metronidazole, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-metronidazole combination cream in tubes or other delivery system.

### Example 2

### Preparation of a Tapinarof-Metronidazole-Phenylephrine Rosacea Cream

Combination composition comprising from about 0.1% w/w to about 2.0% w/w tapinarof, from about 0.25% w/w to about 2.0% w/w metronidazole and from about 0.1% to about 0.5% w/w phenylephrine, wherein phenylephrine is optionally encapsulated.

The topical tapinarof-metronidazole-phenylephrine combination cream consists of:

0.1%-2.0% w/w tapinarof,

0.25%-2.0% w/w metronidazole,

0.1-0.5% w/w phenylephrine,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH,

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, metronidazole, phenylephrine, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-metronidazole-phenylephrine combination cream in a tube or other delivery system.

1. A topical composition comprising a therapeutically effective amount of at least one acaricidal active agent, a therapeutically effective amount of at least one essentially non-acaricidal anti-inflammatory active agent and a carrier suitable for topical administration.

2. The composition of claim 1, wherein the acaricidal active agent is selected from ivermectin, permethrin, niclosamide, benzoyl peroxide, tapinarof and combinations thereof.

3. The composition of claim 1, wherein the essentially non-acaricidal anti-inflammatory active agent is selected from a tetracycline, minocycline, doxycycline, metronidazole, azelaic acid and combinations thereof.

4. The composition of claim 1, wherein the acaricidal active agent is from about 1% to about 12% benzoyl peroxide and the essentially non-acaricidal anti-inflammatory active agent is from about 1% to about 8% w/w minocycline, wherein said composition is optionally formulated as two separate compositions, the first composition comprising from about 1% to about 12% benzoyl peroxide and a carrier suitable for topical administration and the second composition comprising from about 1% to about 8% w/w minocycline and a carrier suitable for topical administration.

5. The composition of claim 1, wherein the acaricidal active agent is from about 0.1% w/w to about 2.0% w/w tapinarof and the essentially non-acaricidal anti-inflammatory active agent is from about 0.25% w/w to about 2.0% w/w metronidazole.

6. The composition of claim 1, wherein the acaricidal active agent is from about 1% w/w to about 12.0% w/w benzoyl peroxide and the essentially non-acaricidal anti-inflammatory active agent is from about 10.0% w/w to about 16.0% w/w azelaic acid.

7. The composition of claim 1, wherein the acaricidal active agent is from about 1% w/w to about 6.0% w/w permethrin and the essentially non-acaricidal anti-inflammatory active agent is from about 10.0% w/w to about 16.0% w/w azelaic acid.

8. The composition of claim 1, further comprising a vasoconstrictor selected from phenylephrine, oxymetazoline, brimonidine, a NOS inhibitor, tetrahydrozoline, pseudoephedrine and combinations thereof, wherein the vasoconstrictor is optionally encapsulated.

9. A dosage form comprising the composition of claim 1, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch and an application syringe.

10. A method of treatment, prevention or alleviation of rosacea by topical administration to the affected skin area of a subject in need thereof a therapeutically effective amount of the composition of claim 1.

11. The method of claim 10, wherein the treatment comprises once daily or twice daily topical administration to the affected skin area of a subject in need thereof of a therapeutically effective amount of the composition.

12. The method of claim 10, wherein the treatment comprises topical administration to the affected skin area of a subject in need thereof of a composition comprising-from about 1% to about 12% benzoyl peroxide and from about 1% to about 8% w/w minocycline, wherein said composition is optionally formulated as two separate compositions, the first composition comprising from about 1% to about 12% benzoyl peroxide and a carrier suitable for topical administration and the second composition comprising from about 1% to about 8% w/w minocycline and a carrier suitable for topical administration, wherein said two separate compositions are administered from two application syringes or a dual chamber application syringe, simultaneously or sequentially in either order.

13. The method of claim 10, wherein the at least one acaricidal active agent and the at least one essentially non-acaricidal anti-inflammatory active agent in said composition exhibit an additive or synergistic effect, thereby allowing to reduce the amounts of the active agents in the composition.

14. A regimen of administration comprising once daily or twice daily administration to the rosacea affected skin area of a subject in need thereof of a therapeutically effective amount of the dosage form of claim 9 until remission.

15. A kit comprising one or more dosage forms of claim 9 and instructions for use.
